(19) United States
(12) Patent Application Publication
(10) Pub. No.: US 2023/0355221 A1
(43) Pub. Date: Nov. 9, 2023

(54) SYSTEMS AND METHOD FOR TRANS-LUMINAL INTRODUCTION OF A MEDICAL DEVICE

(71) Applicant: EndoQuest Robotics, Inc., Houston, TX (US)

(72) Inventors: Dongsuk Shin, Houston, TX (US); Jiwon Choi, Houston, TX (US); Yongman Park, Houston, TX (US); Jeihan Lee, Houston, TX (US); Byeonghui Kim, Seoul (KR)

(73) Assignee: EndoQuest Robotics, Inc., Houston, TX (US)

(21) Appl. No.: 18/221,037

(22) Filed: Jul. 12, 2023

Related U.S. Application Data
(63) Continuation of application No. PCT/US2023/011644, filed on Jan. 26, 2023.
(60) Provisional application No. 63/305,454, filed on Feb. 1, 2022.

(51) Int. Cl. A61B 17/00

(52) U.S. Cl. CPC A61B 17/00234; A61B 2017/00336; A61B 2017/00305; A61B 34/30

(57) ABSTRACT

A method can include inserting a first device into a body lumen having a bend, sliding a first sheath having a first sheath diameter over first device, sliding a second sheath having a second sheath diameter larger than the first sheath diameter over the first device, and navigating the first device through and beyond the bend. The method can also include at least partially straightening the first device to at least partially straighten the bend, removing the first device and the first sheath from the body lumen, leaving the second sheath in place within the at least partially straightened bend, and introducing a second device into the second sheath to navigate the second device through the at least partially straightened bend to an opening of the second sheath.

### CROSS-REFERENCE TO RELATED APPLICATIONS

This application is a continuation of International Patent Application No. PCT/US2023/011644 filed on Jan. 26, 2023, which claims priority to and the benefit of U.S. Provisional Application No. 63/305,454, filed Feb. 1, 2022, the entire contents of which are herein incorporated by reference in their entirety.

### FIELD

This disclosure relates to surgical apparatuses, systems and procedures for minimally invasive surgery. More specifically, the present disclosure is related to surgical apparatuses, systems and procedures for controlling, navigating and deploying medical devices to a targeted site.

### BACKGROUND

In certain transluminal procedures, certain devices can be utilized that are at least partially flexible and/or steerable. Traditionally, the larger the device, the less flexible the device. Certain body lumen (e.g., the colon) define winding pathways with sharp turns and/or multiple turns, presenting difficulty navigating such lumen with less compliant devices. Thus, such lumen can limit the size and type of device that can be used for a transluminal procedure at or beyond the location of the winding pathways.

Such conventional methods and systems have generally been considered satisfactory for their intended purpose. However, there is still a need in the art for improved systems and methods. The present disclosure provides a solution for this need.

### SUMMARY

In accordance with at least one aspect of this disclosure, a method for deploying a medical device can include inserting a first device into a body lumen having a bend, sliding a first sheath having a first sheath diameter over first device, sliding a second sheath having a second sheath diameter larger than the first sheath diameter over the first device, and navigating the first device through and beyond the bend. The method can also include at least partially straightening the first device to at least partially straighten the bend, removing the first device and the first sheath from the body lumen, leaving the second sheath in place within the at least partially straightened bend, and introducing a second device into the second sheath to navigate the second device through the at least partially straightened bend to an opening of the second sheath.

In certain embodiments, sliding the first sheath over the first device can be done after at least partially straightening the first device and before removing the first device from the body lumen. In certain embodiments, sliding the second sheath over the first sheath can be done after sliding the first sheath over the at least partially straightened first device and before removing the first device from the body lumen.

In certain embodiments, the first sheath and the second sheath can be slid over the first device together. In certain embodiments, removing the first device can include removing the first device and the first sheath together or successively. In certain embodiments, inserting the first device can include inserting the first device with the first sheath thereon, and with the second sheath on the first sheath.

In certain embodiments, the second device can be less compliant and/or less steerable than the first device. A diameter of the second device can be larger than a diameter of the first device, for example. In certain embodiments, the diameter of the first device can be about 13 mm or smaller, and the diameter of the second device is about 18 mm or larger. Any suitable relative sizes are contemplated herein.

In certain embodiments, the first device can be an endoscope configured to fit within the first sheath. In certain embodiments, the second device can be or include an overtube configured to fit within the second sheath.

In certain embodiments, navigating the bend can include positioning the opening of the second sheath at or near a target site. In certain embodiments, the method can include extending the second device from the second sheath to the target site. In certain embodiments, the method can include inserting one or more robotic surgical tools through the second device to extend from the second device for access to the target site. Any other suitable procedure is contemplated herein.

In certain embodiments, the body lumen is a colon. For example, the bend can be the sigmoid colon. Any other suitable body lumen or bend thereof is contemplated herein.

In accordance with at least one aspect of this disclosure, a method for navigating a medical device can include navigating an overtube through a prepositioned outer sheath to extend from the outer sheath toward a target site in an intestinal location beyond the sigmoid colon. In certain embodiments, the method can also include prepositioning the outer sheath to traverse the sigmoid colon with a colonoscope and a colonoscope sheath, at least partially straightening the sigmoid colon and the outer sheath with the colonoscope, and removing the colonoscope and colonoscope sheath to leave the outer sheath prepositioned for insertion of the overtube. In certain embodiments, the method can include inserting one or more robotic surgical tools through the overtube to extend from the overtube to access the target site. In certain embodiments, the method can include performing a robotic surgical procedure with the one or more robotic tools at the target site.

In accordance with at least one aspect of this disclosure, a system for deploying a medical device can include an endoscope having an endoscope diameter, the endoscope being configured to navigate through and beyond a bend of a body lumen, and an overtube having an overtube outer diameter larger than the endoscope diameter. The overtube may not be configured to navigate through and beyond the bend of the body lumen until the bend is at least partially straightened. The system can also include a first sheath having a first sheath inner diameter sized to be complimentary to the endoscope outer diameter, and a first sheath outer diameter. The system can also include a second sheath having a second sheath inner diameter sized to be complimentary to both the first sheath outer diameter and complimentary to the overtube outer diameter. In certain embodiments, the endoscope diameter can be about 13 mm or less and the overtube diameter is about 18 mm or larger.

These and other features of the embodiments of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, embodiments thereof will be described in detail herein below with reference to certain figures, wherein:

FIG. 1 shows an embodiment of a method in accordance with this disclosure;

FIG. 2A illustrates a portion of an embodiment of a method in accordance with this disclosure, wherein the method includes navigating a sigmoid colon with a colonoscope;

FIG. 2B illustrates a portion of a method in accordance with this disclosure, wherein the method includes at least partially straightening the sigmoid colon with the colonoscope, e.g., such that the sigmoid colon is telescoped on the colonoscope;

FIG. 2C illustrates a portion of a method in accordance with this disclosure, wherein the method includes inserting a first sheath and a second sheath over the colonoscope;

FIG. 2D shows a cross-section of the structure of FIG. 2C within the body lumen, having a first sheath and a second sheath over the colonoscope;

FIG. 2E illustrates a portion of a method in accordance with this disclosure, wherein the method includes removing the first sheath and colonoscope (e.g., simultaneously, or colonoscope first, for example) leaving the second sheath in place;

FIG. 2F shows a cross-section of the remaining structure of FIG. 2E within the body lumen, having only the second sheath;

FIG. 2G illustrates a portion of a method in accordance with this disclosure, wherein the method includes inserting an overtube having a plurality of lumen into the second sheath (e.g., the overtube having a video scope disposed therein during insertion);

FIG. 2H shows a cross-section of the structure of FIG. 2E within the body lumen, having the second sheath around the overtube;

FIG. 3A illustrates a portion of an embodiment of a method in accordance with this disclosure, wherein an endoscope (e.g., a colonoscope) having a first sheath and second sheath thereon is inserted into a body lumen (e.g., a sigmoid colon);

FIG. 3B illustrates a portion of an embodiment of a method in accordance with this disclosure, wherein the endoscope is navigated toward a target site;

FIG. 3C illustrates a portion of an embodiment of a method in accordance with this disclosure, wherein the endoscope is positioned at or near the target site;

FIG. 3D illustrates a portion of an embodiment of a method in accordance with this disclosure, wherein the first sheath and the second sheath are advanced over the endoscope toward the target site while the endoscope is positioned at or near the target site such that a distal end of the second sheath is at or near the target site;

FIG. 3E illustrates a portion of an embodiment of a method in accordance with this disclosure, wherein the first sheath and the endoscope are removed leaving behind the second sheath having a distal end that is positioned at or near the target site;

FIG. 3F illustrates a portion of an embodiment of a method in accordance with this disclosure, wherein an overtube is inserted and advanced through the second sheath to be positioned at or near the target site;

FIG. 3G illustrates a portion of an embodiment of a method in accordance with this disclosure, wherein a plurality of instruments (e.g., configured to be robotically controlled) are advanced through the overtube to extend from the overtube be positioned at or near the target site to be able to act on the target site; and

FIG. 3H is a perspective view of the distal end of the assembly of FIG. 3G shown positioned at the target site, shown having the overtube, a videoscope extending through and from the overtube, and two instruments extending through and from the overtube.

### DETAILED DESCRIPTION

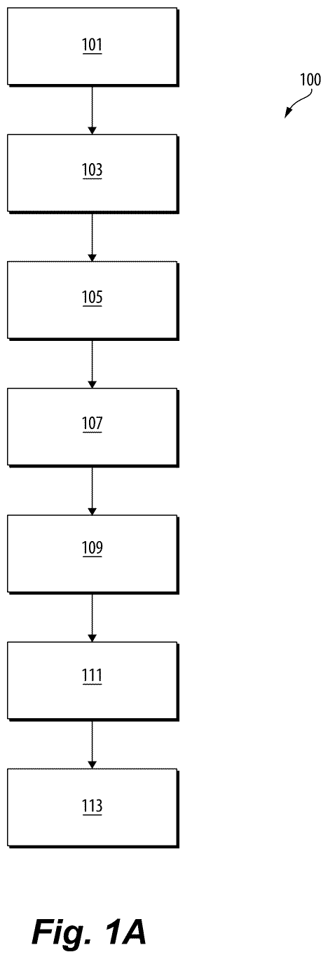

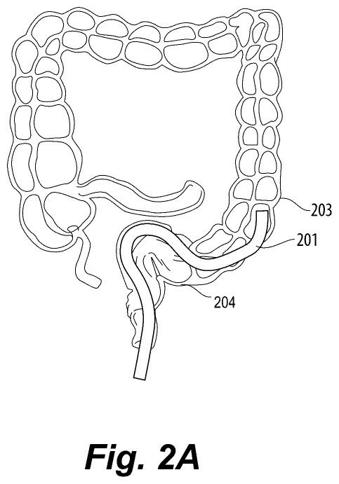

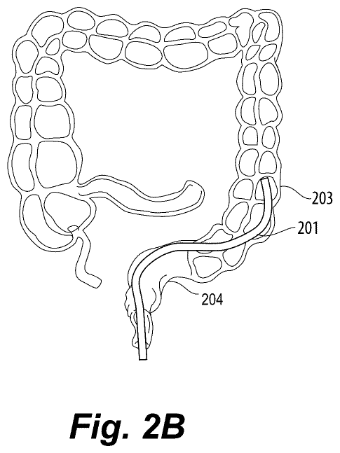

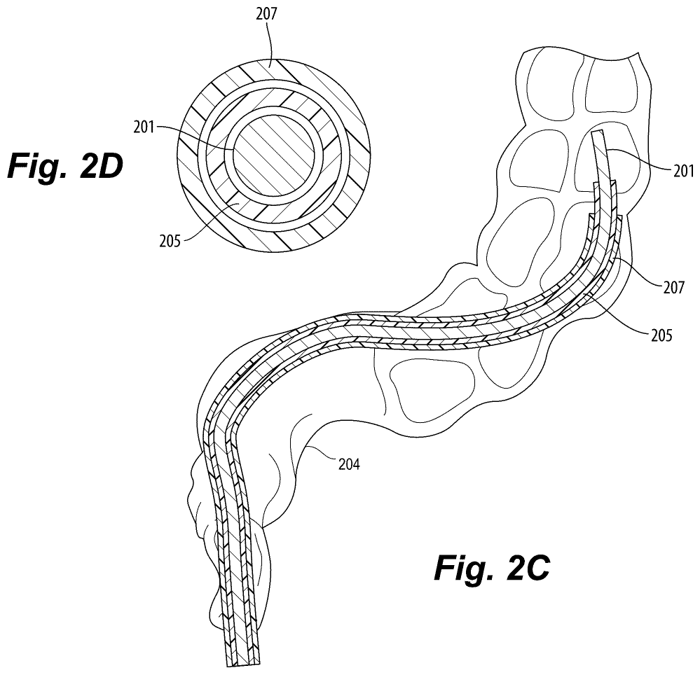

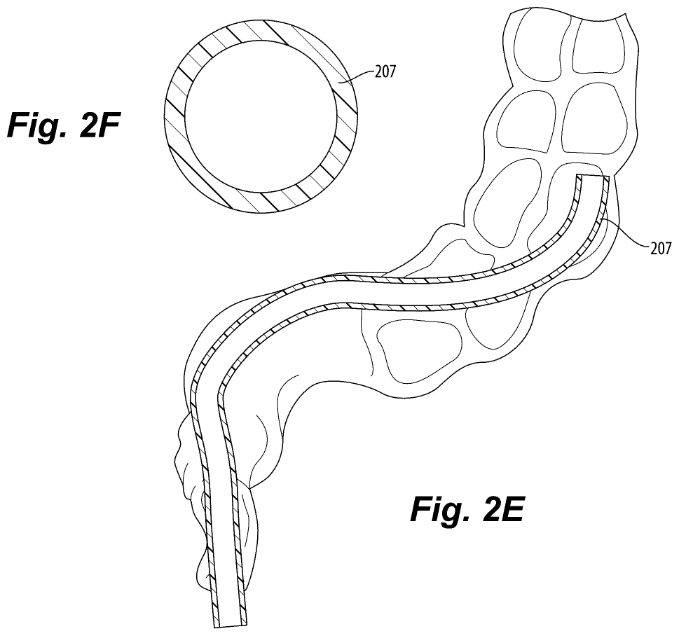

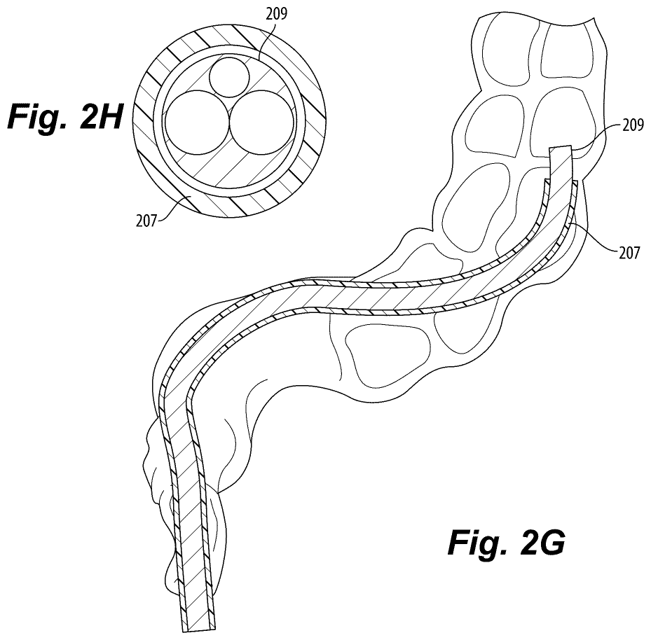

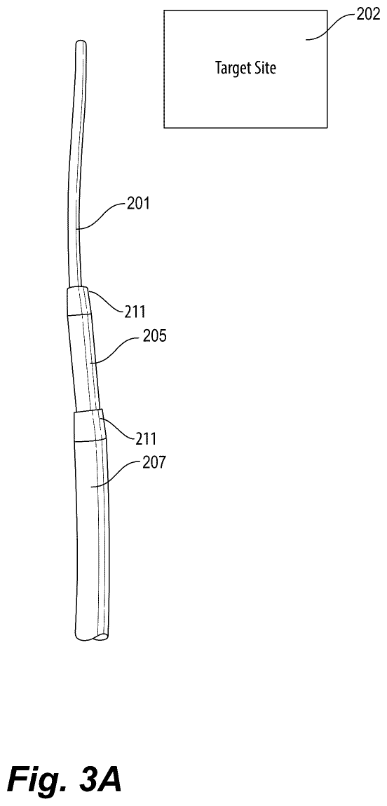

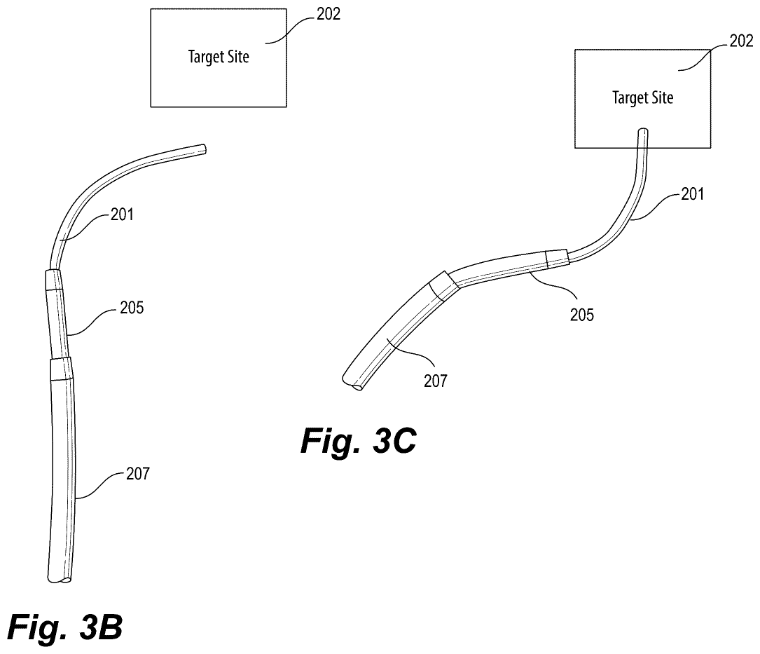

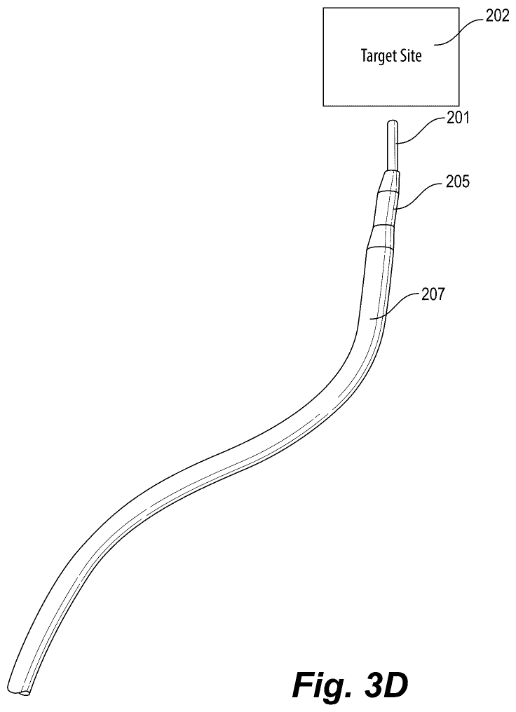

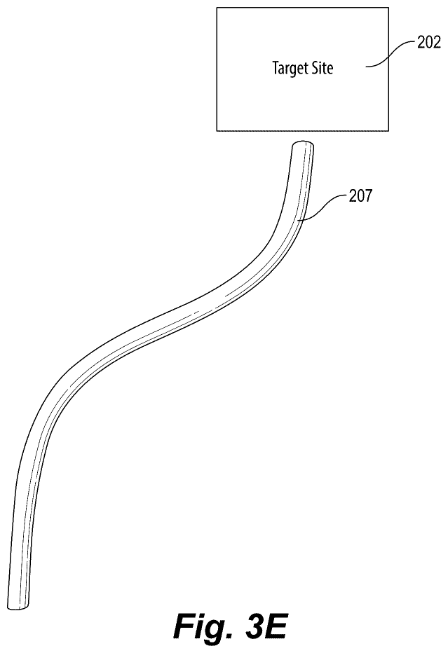

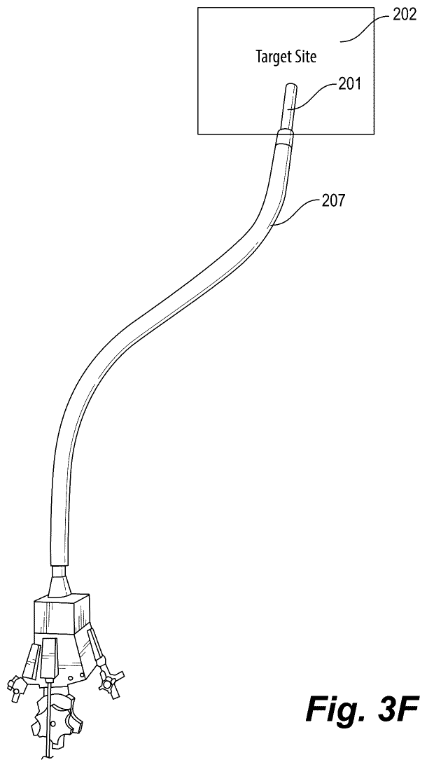

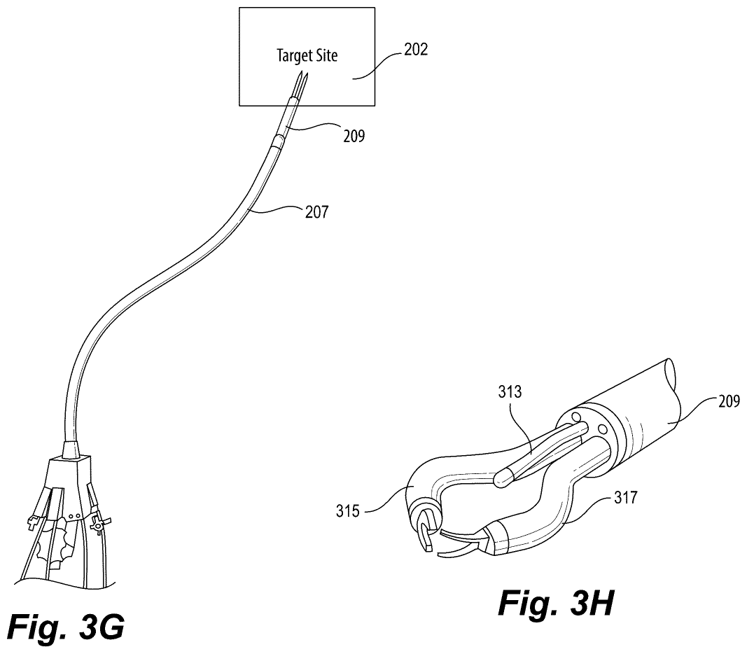

Reference will now be made to the drawings wherein like reference numerals identify similar structural features or aspects of the subject disclosure. For purposes of explanation and illustration, and not limitation, an illustrative view of an embodiment of a method in accordance with the disclosure is shown in FIG. 1 and is designated generally by reference character 100. Other embodiments and/or aspects of this disclosure are shown in FIGS. 2A-3H.

In accordance with at least one aspect of this disclosure, referring to the embodiments shown in FIGS. 1 and 2A-2H, a method 100 can include inserting, e.g., at block 101, a first device 201 (e.g., an endoscope such as colonoscope) into a body lumen 203 (e.g., a colon or any other suitable body lumen) having a bend 204 (e.g., the sigmoid colon, splenic flexure, and/or any other suitable bend). The method 100 can include sliding, e.g., at block 103, a first sheath 205 having a first sheath diameter over first device 201, and sliding, e.g., at block 105, a second sheath 207 having a second sheath diameter larger than the first sheath diameter over the first device 201. The method 100 can include navigating, e.g., at block 107, the first device 201 through and beyond the bend.

The method 100 can also include at least partially straightening, e.g., at block 109, the first device 201 to at least partially straighten the bend. In certain embodiments, straightening certain body lumen 203 (e.g., a sigmoid colon for example) can cause telescoping of the tissue (e.g., folding of the tissue onto itself such as a bellows), also referred to as intussusception.

The method 100 can include removing, e.g., at block 111, the first device 201 and the first sheath 205 from the body lumen 203, leaving the second sheath 207 in place within the at least partially straightened bend. The method 100 can include introducing, e.g., at block 113, a second device 209 into the second sheath 207 to navigate the second device 209 through the at least partially straightened bend to an opening of the second sheath 207. The second sheath 207 can have suitable rigidity to maintain the body lumen 203 in the at least partially straightened position such that when the first device 201 and the first sheath 205 are removed, the body lumen 203 is maintained suitably straight for insertion of the second device 209.

In certain embodiments, as shown in FIG. 2B, sliding the first sheath 205 over the first device 201 can be done after at least partially straightening the first device 201 and before removing the first device 201 from the body lumen 203. In certain embodiments, sliding the second sheath 207 over the first sheath 205 can be done after sliding the first sheath 205 over the at least partially straightened first device 201 and before removing the first device 201 from the body lumen 203.

In certain embodiments, the first sheath 205 and the second sheath 207 can be slid over the first device 201 together. In certain embodiments, removing the first device 201 can include removing the first device 201 and the first sheath 205 together or successively. In certain embodiments, inserting the first device 201 can include inserting the first device 201 with the first sheath 205 thereon, and with the second sheath 207 on the first sheath 205, e.g., as shown in FIG. 3A.

In certain embodiments, the second device 209 can be less compliant and/or less steerable than the first device 201. For example, a diameter of the second device 209 can be larger than a diameter of the first device 201, for example. In certain embodiments, the diameter of the first device 201 can be about 13 mm or smaller, and the diameter of the second device 209 is about 18 mm or larger. Any suitable relative sizes are contemplated herein. In certain embodiments, the second device 209 is not capable of steering or flexing through one or more bends of the body lumen 203, while the first device 201 is capable.

In certain embodiments, the first device 201 can be an endoscope configured to fit within the first sheath 205. In certain embodiments, the second device 209 can be or include an overtube (e.g., as shown in FIGS. 2G, and FIGS. 3F-3H) configured to fit within the second sheath 207. In certain embodiments, the length of the first device 201 is longer than the length of the first sheath 205, while the length of the first sheath 205 is longer than the length of the second sheath 207. The first sheath 205 and the second sheath 207 can include a tapered end 211 (e.g. FIG. 3A) formed at distal ends of the first sheath 205 and the second sheath 207, for example. This can assist the second sheath 207 sliding over the first sheath 205 and the first sheath 205 is sliding over the at least partially straightened first device 201 to control traceability (the ability of a medical device to follow the tip around bends), steerability, and torquability, for example.

FIGS. 2A-2H illustrate an embodiment of a method in accordance with this disclosure. FIG. 2A illustrates a portion of an embodiment of a method, which can include navigating a sigmoid colon with a colonoscope. FIG. 2B illustrates a portion of the method which can include at least partially straightening the sigmoid colon with the colonoscope, e.g., such that the sigmoid colon is telescoped on the colonoscope. FIG. 2C illustrates a portion of the method which can include inserting a first sheath and a second sheath over the colonoscope. FIG. 2D shows a cross-section of the structure shown in the method of FIG. 2C within the body lumen, having a first sheath and a second sheath over the colonoscope. FIG. 2E illustrates a portion of the method which can include removing the first sheath and colonoscope (e.g., simultaneously, or colonoscope first, for example) leaving the second sheath in place. FIG. 2F shows a cross-section of the remaining structure of FIG. 2E within the body lumen, having only the second sheath. FIG. 2G illustrates a portion of the method which can include inserting an overtube having a plurality of lumen into the second sheath (e.g., the overtube having a video scope disposed therein during insertion). FIG. 2H shows a cross-section of the structure resulting from the method of FIG. 2E within the body lumen, having the the second sheath around the overtube.

FIGS. 3A-3H illustrate an embodiment of a method in accordance with this disclosure. FIG. 3A illustrates an endoscope (e.g., a colonoscope) having a first sheath and second sheath thereon being inserted into a body lumen (e.g., a sigmoid colon). FIG. 3B illustrates the endoscope being navigated toward a target site. FIG. 3C illustrates the endoscope being positioned at or near the target site. FIG. 3D illustrates the first sheath and the second sheath being advanced over the endoscope toward the target site while the endoscope is positioned at or near the target site such that a distal end of the second sheath is at or near the target site. FIG. 3E illustrates the first sheath and the endoscope being removed leaving behind the second sheath having a distal end that is positioned at or near the target site. FIG. 3F illustrates an overtube being inserted and advanced through the second sheath to be positioned at or near the target site. FIG. 3G illustrates a plurality of instruments (e.g., configured to be robotically controlled) are advanced through the overtube to extend from the overtube be positioned at or near the target site to be able to act on the target site. FIG. 3H is a perspective view of the distal end of the assembly of FIG. 3G shown positioned at the target site, shown having the overtube 209, a videoscope 313 extending through and from the overtube 209, and two instruments 315, 317 extending through and from the overtube 209.

As shown in FIGS. 3B-3H, in certain embodiments, navigating the bend can include positioning the opening of the second sheath 207 at or near a target site 202 (e.g., a site for robotic surgery). In certain embodiments, the method can include extending the second device 209 from the second sheath 207 to the target site 202. In certain embodiments, the method can include inserting one or more robotic surgical tools through the second device 209 to extend from the second device 209 for access to the target site 202. Any other suitable procedure is contemplated herein.

In certain embodiments, the body lumen 203 is a colon. For example, the bend can be the sigmoid colon. Any other suitable body lumen or bend (e.g., the splenic flexure) thereof is contemplated herein.

In accordance with at least one aspect of this disclosure, a method can include navigating an overtube through a prepositioned outer sheath to extend from the outer sheath toward a target site in an intestinal location beyond the sigmoid colon. In certain embodiments, the method can also include prepositioning the outer sheath to traverse the sigmoid colon with a colonoscope and a colonoscope sheath, at least partially straightening the sigmoid colon and the outer sheath with the colonoscope, and removing the colonoscope and colonoscope sheath to leave the outer sheath prepositioned for insertion of the overtube. In certain embodiments, the method can include inserting one or more robotic surgical tools through the overtube to extend from the overtube to access the target site. In certain embodiments, the method can include performing a robotic surgical procedure with the one or more robotic tools at the target site.

In accordance with at least one aspect of this disclosure, a system can include an endoscope (e.g., device 201) having an endoscope diameter, the endoscope being configured to navigate through and beyond a bend of a body lumen. The system can include an overtube (e.g., overtube 209) having an overtube outer diameter larger than the endoscope diameter. The overtube (e.g., overtube 209) may not be configured to navigate through and beyond the bend of the body lumen until the bend is at least partially straightened. The system can also include a first sheath 205 having a first sheath inner diameter sized to be complimentary to the endoscope outer diameter, and a first sheath outer diameter. The system can also include a second sheath 207 having a second sheath inner diameter sized to be complimentary to both the first sheath outer diameter and complimentary to the overtube outer diameter. In certain embodiments, the endoscope diameter can be about 13 mm or less and the overtube diameter is about 18 mm or larger. The overtube 209 can be any suitable overtube, e.g., a flexible elongated insertion tube or steerable shaft having one or more instrument channels therein for the medical device to advance/retract therein, which can be controlled manually and/or robotically. For example, in certain embodiments, the overtube can be an overtube as disclosed in commonly owned PCT application no. PCT/US2022/051259 filed Nov. 29, 2022, which is incorporated by reference herein in its entirety.

While embodiments of a method are disclosed herein in a certain literal order, or a certain shown order, the method(s) can be carried out in any suitable order (e.g., as ordered literally, as shown, or otherwise) to achieve a desired result (e.g., introduction of the second device to the target site past a bend in the body lumen), and are not limited to the orders literally described or shown. For example, the order of actions in the embodiments disclosed herein can be as shown, or can be any other suitable order (e.g., such that the second sheath remains in the at least partially straightened body lumen before the second device is introduced). For example, the first sheath and second sheath can be slid on before straightening the first device as described above with respect to FIG. 1, or after straightening the first device as shown in the embodiment of FIGS. 2A-2H. Also, as shown in FIGS. 3A-3H, the first sheath and the second sheath can be disposed on the endoscope prior to insertion of the endoscope.

While embodiments are shown having certain portions, embodiments can include any other suitable method(s) and/or portion(s) thereof. Any suitable additional or alternative steps are contemplated herein.

Embodiments can be used to provide robotic surgery within any lumen of the body, regardless of complex bends of the lumen. For example, procedures within the colon beyond the sigmoid colon are made possible. Embodiments can allow robotic surgery transluminally inserted through the rectum to target sites within the sigmoid colon, the descending colon, the splenic flexure, and/or the transverse colon, for example.

Embodiments can include a dual sheath arrangement. For example, a traditional colonoscope can be smaller than an overtube, and thus both types of sheaths can be used to allow the removal of the colonoscope and insertion of the overtube.

Embodiments of a method can include using smaller diameter colonsocope with two sheaths to navigate the sigmoid colon, then straightening, then pulling out smaller diameter colonoscope and first overtube components to leave second, e.g., outer, sheath in place straightened, then inserting larger diameter robotic surgery overtube into the straightened channel. While embodiments are disclosed using an endoscope as a straightening device, any suitable straightening device is contemplated herein.

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the subject disclosure includes reference to certain embodiments, those skilled in the art will readily appreciate that changes and/or modifications may be made thereto without departing from the spirit and scope of the subject disclosure.

What is claimed is:

1. A method for deploying a medical device, comprising:
inserting a first device into a body lumen having a bend;
sliding a first sheath having a first sheath diameter over first device;
sliding a second sheath having a second sheath diameter larger than the first sheath diameter over the first device;
navigating the first device through and beyond the bend;
at least partially straightening the first device to at least partially straighten the bend;
removing the first device and the first sheath from the body lumen, leaving the second sheath in place within the at least partially straightened bend; and
introducing a second device into the second sheath to navigate the second device through the at least partially straightened bend to an opening of the second sheath.

2. The method of claim 1, wherein sliding the first sheath over the first device is done after at least partially straightening the first device and before removing the first device from the body lumen.

3. The method of claim 2, wherein sliding the second sheath over the first sheath is done after sliding the first sheath over the at least partially straightened first device and before removing the first device from the body lumen.

4. The method of claim 2, wherein the first sheath and the second sheath are slid over the first device together.

5. The method of claim 3, wherein removing the first device includes removing the first device and the first sheath together or successively.

6. The method of claim 1, wherein inserting the first device includes inserting the first device with the first sheath thereon, and with the second sheath on the first sheath.

7. The method of claim 1, wherein the second device is less compliant and/or less steerable than the first device.

8. The method of claim 7, wherein a diameter of the second device is larger than a diameter of the first device.

9. The method of claim 8, wherein the diameter of the first device is about 13 mm or smaller, and the diameter of the second device is about 18 mm or larger.

10. The method of claim 8, wherein the first device is an endoscope configured to fit within the first sheath.

11. The method of claim 10, wherein the second device is or includes an overtube configured to fit within the second sheath.

12. The method of claim 1, wherein navigating the bend includes positioning the opening of the second sheath at or near a target site.

13. The method of claim 12, further comprising extending the second device from the second sheath to the target site.

14. The method of claim 13, further comprising inserting one or more robotic surgical tools through the second device to extend from the second device for access to the target site.

15. The method of claim 1, wherein the body lumen is a colon, wherein the bend is the sigmoid colon.

16. A method for navigating a medical device, comprising:
navigating an overtube through a prepositioned outer sheath to extend from the outer sheath toward a target site in an intestinal location beyond the sigmoid colon.

17. The method of claim 16, further comprising:
prepositioning the outer sheath to traverse the sigmoid colon with a colonoscope and a colonoscope sheath;
at least partially straightening the sigmoid colon and the outer sheath with the colonoscope; and
removing the colonoscope and colonoscope sheath to leave the outer sheath prepositioned for insertion of the overtube.

18. The method of claim 17, further comprising inserting one or more robotic surgical tools through the overtube to extend from the overtube to access the target site.

19. The method of claim 18, further comprising performing a robotic surgical procedure with the one or more robotic tools at the target site.

20. A system for deploying a medical device, comprising:
an endoscope having an endoscope diameter, the endoscope being configured to navigate through and beyond a bend of a body lumen;
an overtube having an overtube outer diameter larger than the endoscope diameter, wherein the overtube is not configured to navigate through and beyond the bend of the body lumen until the bend is at least partially straightened;
a first sheath having a first sheath inner diameter sized to be complimentary to the endoscope outer diameter, and a first sheath outer diameter; and
a second sheath having a second sheath inner diameter sized to be complimentary to both the first sheath outer diameter and complimentary to the overtube outer diameter.
